(19) United States
(12) Patent Application Publication
(10) Pub. No.: US 2025/0360239 A1
(43) Pub. Date: Nov. 27, 2025

(54) Radiation-Based In-Situ Sterilization for Sample Return Missions

(71) Applicant: G21 Nucleonics Inc., Seattle, WA (US)

(72) Inventor: Christopher G. Morrison, Seattle, WA (US)

(73) Assignee: G21 Nucleonics Inc., Seattle, WA (US)

(21) Appl. No.: 19/216,150

(22) Filed: May 22, 2025

Related U.S. Application Data
(60) Provisional application No. 63/650,925, filed on May 23, 2024.

(51) Int. Cl. A61L 2/08; A61L 2/04; A61L 2/10; A61L 2/232; A61L 2/238; A61L 2/26; G01N 1/44; G21F 5/015

(52) U.S. Cl. CPC A61L 2/087; A61L 2/04; A61L 2/081; A61L 2/082; A61L 2/10; A61L 2/232; A61L 2/238; A61L 2/26; G01N 1/44; G21F 5/015; A61L 2202/11; A61L 2202/121; A61L 2202/122

(57) ABSTRACT

The present disclosure teaches methods and devices for using radioisotope sources to sterilize samples of soil, rock, atmosphere or atmosphere, which may be collected from an extraterrestrial planet, moon, or asteroid, or other body. A sterilization method may include: (a) providing a sample container, (b) inserting a sample collection tube, containing a sample, into the sample container, (c) inserting a radioisotope source into the sample container, and (d) irradiating the sample with radiation emitted by the radioisotope source. A sterilization apparatus may include: a sample container, a sample collection tube, a sample disposed inside of the sample collection tube, and a radioisotope source disposed inside of the sample container. Alternatively, an outside surface of the sample container may be irradiated with radiation emitted by a radioisotope source that is attached, or coated onto, the outside surface of the sample container. The radioisotope source may be Cesium-137, Am-241, or Tl-204, or combinations thereof.

### CROSS-REFERENCES TO RELATED APPLICATIONS

This application claims a priority benefit of U.S. Provisional 63/650,925 filed May 23, 2024, “Radiation-Based In-Situ Sterilization for Sample Return Missions”, which is incorporated herein by reference in its entirety.

### INTRODUCTION

The present disclosure teaches methods and devices for using radioisotope sources to sterilize samples of soil or rock, which may be collected from an extraterrestrial planet, moon, or asteroid.

Scientists are excited about performing soil or rock sample return missions throughout the solar system. Locations such as Mars and the icy moons of the outer planets are prime candidates in the search for life. However, care must be taken to protect Earth's environment from the potential risk of bringing that life back to Earth (i.e., backward contamination) and ensure that Earth life is sterilized and cannot affect potential ecosystems on other planets. Forward and backward contamination between planetary bodies is a paramount challenge for sample return missions. To prevent the biological transfer of possible extraterrestrial microbes, strict rules for planetary protection exist (see, e.g., NASA's NPR 8715.24). Complex sterilization, redundant seal containments, lengthy quarantines, special handling, and mission concept of operations are the current state of the art for planetary protection. In the case of Mars Sample Return, this results in additional mass for the sample return containers, chemical- and heat-based sterilization systems, and ground facilities. Each link in the chain of sample return is consequently heavier and more expensive.

This disclosure describes a novel mission element that revolutionizes the task of planetary protection: a radioisotope-based sterilization system. NASA's extensive history of utilizing radioisotope technology across the Solar System is the foundation of this innovative approach. While space radioisotope systems have traditionally focused on heat and electricity, the present disclosure focuses primarily on the use of ionizing radiation. This form of irradiation sterilization is useful because it directly affects nucleic acid (DNA and RNA associated with viruses and bacteria), while leaving the bulk material unaffected. The use of ionizing radiation is unlike heat or chemical sterilization, which can damage the samples due to chemical transformation and leave behind residue. Prions (non-DNA-based pathogenic proteins) can also be sterilized by radiation. While sterilization typically requires a very intense radiation source for full sterilization in a short period, a sample return mission offers the advantage of having upwards of hundreds of days available for sterilization during the return trip to Earth. This makes a low-activity radiation source a practical and efficient choice over a long travel period for return samples. Placing a small radiation source close to the samples during their journey on the Earth Return Orbiter would provide a straightforward, low-mass, high-efficiency mechanism for robust planetary protection.

### SUMMARY

The present disclosure teaches methods and devices for using radioisotope sources to sterilize samples of soil, rock, atmosphere, or other local material, which may be collected from an extraterrestrial planet, moon, asteroid, or other body. A sterilization method may include: (a) providing a sample container, (b) inserting a sample collection tube, containing a sample, into the sample container, (c) inserting a radioisotope source into the sample container, and (d) irradiating the sample with radiation emitted by the radioisotope source. A sterilization apparatus may include: a sample container, a sample collection tube, a sample disposed inside of the sample collection tube, and a radioisotope source disposed inside of the sample container. Alternatively, an outside surface of the sample container may be irradiated with radiation emitted by a radioisotope source that is attached to, or coated on, an outside surface of the sample container. The radioisotope source may be Cs-137 (Cesium-137), Am-241, or Tl-204, or combinations thereof.

In a first method embodiment, a sterilization method includes: (a) providing a sample container; (b) inserting a sample collection tube, containing a sample, into the sample container; (c) providing a radioisotope source; and (d) irradiating the sample with radiation emitted by the radioisotope source. Step (d) may include irradiating the sample with ionizing radiation at a dose rate of at least 0.001 Gy/s. Step (d) may also include irradiating the sample for a sustained period of at least three years to a total dose of at least 100 kGy.

In some method embodiments, the radioisotope source comprises Cesium-137. The radioisotope source may have an activity of at least 15 Ci of Cesium-137.

In some method embodiments, the radioisotope source is disposed inside of the sample container.

In some method embodiments, the radioisotope source is disposed outside of the sample container.

In a method embodiment, the radioisotope source may be encapsulated in glass. The sample container may be a Mars Sample Return Container. The sterilization method may further include a MARS Capture and Containment Return System (MCCRS); wherein the MCCRS has a mass of less than or equal to about 100 kg.

In a method embodiment, the radioisotope source is centrally located inside of the sample container. The radioisotope source may radiate photons with a photon energy ranging from about 50 keV to about 1000 keV The radioisotope source may be shipped in a Type A container.

In a method embodiment, the method may further include irradiating the sample to a dose sufficient to achieve a Sterilization Assurance Level (SAL) for microbes that ranges between about 10⁻¹² to about 10⁻²⁴.

In a method embodiment, the sterilization method may include: (a) providing a sample container having an outside surface; (b) providing a radioisotope source disposed on the outside surface of the sample container; and (c) irradiating the outside surface of the sample container with radiation emitted by the radioisotope source. The radioisotope source may emit at least one type of radiation selected from the group consisting of (1) low energy Photons with an energy less than 100 keV, (2) Beta particles with an energy ranging from about 300 keV to about 2.5 MeV, and (3) Alpha particles with an energy greater than about 5 MeV, and combinations thereof.

In a method embodiment, the radioisotope source may have at least 4 mCi of Am-241 or at least 1 Ci of Tl-204. The radioisotope source may also emit photons with an energy ranging from 200 to 300 keV; or the radioisotope source may emit photons with a mean free path of less than or equal to about 200 μm in Titanium.

In a method embodiment, the sterilization method may include: applying a radioactive coating to the outside surface of the sample container; wherein the radioactive coating comprises the radioisotope source. A method of applying the radioactive coating may include: electroplating, chemical vapor deposition, physical vapor deposition, cold spraying, or 3-D additive printing the radioisotope source on the outside surface, or combinations thereof. Alternatively, applying the radioactive coating may include adding the radioisotope source to a paint and then depositing the radioactive paint to the outside surface.

In a method embodiment, the sterilization method may include: treating the outside surface with at least two different sterilization methods selected from the group consisting of: illuminating with ultraviolet light, irradiating with Gamma rays, heating to an elevated temperature, and applying a toxic chemical, and combinations thereof.

In a method embodiment, the outside surface of the sample container may be subjected to a combination of three different sterilization methods, including: (1) using an antimicrobial surface finish; (2) illuminating the outside surface with ultraviolet light, and (3) irradiating the outside surface with radiation emitted by one or more radioisotope sources attached to the outside surface.

In a first apparatus embodiment, a sterilization apparatus includes: a sample container; a sample collection tube; a sample disposed inside of the sample collection tube; and a radioisotope source; wherein the sample collection tube is disposed inside of the sample container; and wherein the radioisotope source is disposed inside of the sample container. The sample container may be a hollow cylinder; the radioisotope source may be centrally located inside of the hollow cylinder and the radioisotope source may be positioned adjacent to the sample collection tube.

In an apparatus embodiment, the sample container includes a proximal cylindrical cup and a distal cylindrical cup configured to mate together along a common central axis to form a sealed sample container; wherein the radioisotope source is attached to the proximal cylindrical cup; and wherein the sample collection tube is positioned inside of the distal cylindrical cup.

In an apparatus embodiment, the sterilization apparatus may include a sealed cylindrical radioisotope source tube containing the radioisotope source; wherein the sample container includes a radioisotope source support structure that is configured for holding the sealed cylindrical radioisotope source tube inside of the sample container; and wherein the sample container may further include a sample collection tube support structure that is configured for holding the sample collection tube inside of the sample container. The radioisotope source may include Cesium-137, and it may have at least 15 Ci of Cesium-137. The Cesium-137 radioisotope source may be encapsulated in glass.

In an apparatus embodiment, a sterilization apparatus includes: a sample container having an outside surface; a sample collection tube; a sample disposed inside of the sample collection tube; and a radioisotope source disposed on the outside surface of the sample container; wherein the sample collection tube is located inside of the sample container. The radioisotope source may emit at least one type of radiation selected from the group consisting of low energy Photons with an energy less than 100 keV, Beta particles with an energy ranging from about 300 keV to about 2.5 MeV, and Alpha particles with an energy greater than about 5 MeV; and combinations thereof. The radioisotope source may have at least 4 mCi of Am-241, or it may have at least 1 Ci of Tl-204. The radioisotope source may emit photons with an energy ranging from about 200 keV to about 300 keV.

In an apparatus embodiment, the sample container may be made of titanium; and the radioisotope source may emit photons with a mean free path of less than or equal to about 200 μm in Titanium.

In an apparatus embodiment, an outside surface of the sample container may be coated with antimicrobial copper and/or silver to provide an additional layer of sterilization.

### BRIEF DESCRIPTION OF THE DRAWINGS

FIG. 1A shows a graph illustrating a theoretical logarithmic-linear relationship between a radiation dose and the number of surviving microorganisms (i.e., survival fraction).

FIG. 1B shows a graph illustrating a non-linear, empirical relationship between radiation dose and microorganism survival fraction for several strains of microbes.

FIG. 2 shows the radioactive decay scheme for Cesium-137 (Cs-137).

FIG. 3 shows a perspective, 3-D, cut-away view of an example of a sample collection tube.

FIG. 4 shows an exploded side view of an example of a sample container with multiple, in-situ, radioisotope sterilization sources and multiple sample collection tubes contained within the sample container.

FIG. 5 shows a cross-sectional, end view (SEC. A-A) of the sample container illustrated previously in FIG. 4.

FIG. 6 shows a perspective, 3-D view of an example of sample container with multiple, in-situ radioisotope sterilization sources and multiple sample collection tubes contained within the sample container.

FIG. 7 shows a graph illustrating the Range of particle deposition (mm) for energetic Alpha or Beta particles, and also the Mean Free Path (MFP) deposition distance for energetic photons, in Titanium, as a function of particle energy (keV).

### DETAILED DESCRIPTION OF THE DISCLOSURE

The sterilization devices disclosed herein may be used for sterilizing DNA, RNA, and protein-based pathogens. These sterilization devices may be used for sterilizing living organisms such as bacteria, fungi, and other single or muti-celled life, as well as non-living pathogens such as viruses and prions. These pathogens may be contained within soil samples collected from extraterrestrial sources, such as planets (e.g., Mars or Jupiter), asteroids, etc.

The term “microbe” means a microorganism, which includes singled celled and muti-celled organisms (including insects and worms), prions, and viruses. The term “sample” refers to both samples of extraterrestrial soil and rock samples. The terms “Ci” and “Curie” have the same meaning. The terms “radioisotope source” and “radioactive source” are used interchangeably. The term “about” means +/−10% of a referenced value.

### Sterilization Criteria

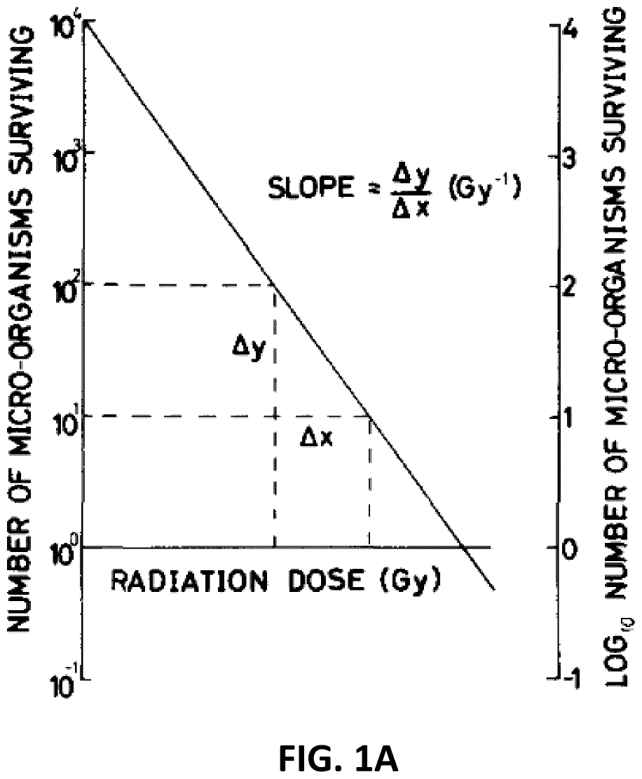

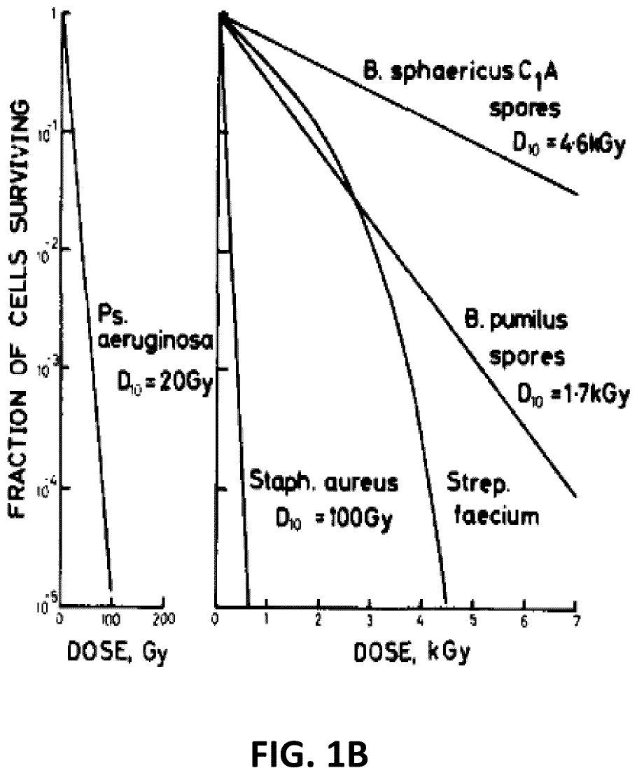

FIG. 1A shows a graph illustrating a theoretical logarithmic-linear relationship between the radiation dose and the number of surviving microorganisms (i.e., survival fraction). A useful metric is the sterilization assurance level (SAL). SAL measures the probability of a viable microorganism surviving after being sterilized. In the medical field, a SAL of 10⁻⁶ is medically defined as sterile and indicates a one-in-one-million chance of leaving a viable microbe. The radiation dose necessary to sterilize a sample differs for various entities, such as bacteria and viruses [1]. As shown in FIG. 1, the relationship between radiation dose and SAL is non-linear; doubling the dose will exponentially reduce the number of surviving microbes.

FIG. 1B shows a graph illustrating a non-linear, empirical relationship between radiation dose and microorganism survival fraction for several strains of microbes.

As a microbe is irradiated, there is a slope on a linear-log plot that describes survivability. The slope of that line depends on the microbe strain and the temperature at which the microbe is irradiated. At low doses, some microbes have resistance to radiation due to their unique biology (for example, the ability to repair a single strand break); however at high doses (on the order of kGy or more) the trend follows the line as the biological repair and resistance mechanisms are unable to compensate for the number of DNA and RNA breaks (this is known as breaking the “shoulder” of the curve). Protein-based prions have also been shown to be sterilized by radiation with doses of 25-50 kGy [19]. The SAL is generally unaffected by different types of radiation or dose rates. Hansen et al. (2020) [3] state in their sensitivity study of radiation types that: “Based on analysis of the data, no significant differences were seen in the rate of microbial lethality across the range of radiation energies evaluated. In summary, as long as proof exists that the specified dose is delivered, dose is dose.” [3]

Hansen et al. [3] evaluated dose rates between 36 MGy/h and 370 Gy/h (0.1 Gy/s to 10 kGy/s). Extrapolating to lower dose rates may require additional study. At a low enough dose rate, DNA can be repaired over time. However, repair requires energy, and organisms would likely be oligotrophic and unable to sustain repair. An example of a non-limiting, dose rate proposed in this disclosure is about 0.001 Gy/s (e.g., 100 kGy over 3 years). However, a higher dose rate may be used, if necessary.

A dose delivered over hundreds of days will be just as effective as a dose delivered in a few minutes. This is a key insight as a sample return mission takes hundreds of days to return to Earth. The radioisotope inventory can be decreased by five to six orders of magnitude, reducing from a kCi scale needed in high-throughput terrestrial facilities to mCi scale sources for a sample return mission. This significant reduction in radioisotope inventory does not compromise the effectiveness of the sterilization process, ensuring planetary protection guidelines and regulations are adhered to.

For a sample return mission, a question becomes: what is the appropriate dose to ensure no backward contamination. A dose of 25 kGy is internationally accepted for medical sterilization, providing a SAL of 10⁻⁶ (Kowalski & Tallentire, 1999). Due to the uncertainty of the nature of possible life, it would be wise to apply a conservative Factor of Safety (FoS) on the dose to ensure no reasonable doubt of complete sterilization. A conservative FoS between 2 and 4 (resulting in a dose of 50-100 kGy) would follow the line described in FIG. 1A. While we don't know the slope of the line for possible Martian life, for the hardiest Earth microbes, this would have a SAL of 10⁻¹² to 10⁻²⁴ corresponding to the 50 kGy to 100 kGy dose (or 5-10 MRad). Even if Martian life was more resistant to radiation, this makes a compelling case for sterilization. This FoS may be weighed against the desire to preserve the maximum scientific value of any sterilized biological material in the sample and engineering concerns related to a radioisotope.

### Sample Integrity

There are many reasons for studying a sample, including geological, industrial, extant life, and extinct life. Each discipline has different criteria for sample integrity. The Mars scientific community is diverse, and some information is available on sample integrity requirements [20].

While radioisotope sterilization can be achieved with a sufficiently large dose, the integrity of the sample for its end use should not be affected by radiation. Radiation is a capable sterilization process because it can easily travel through membranes and layers and randomly targets atoms. Other sterilization mechanisms, such as heat and chemicals, may break down protective layers to kill cells, which may be more destructive. Long molecules, specifically nucleic acids, are especially susceptible to radiation since they are at least hundreds of millions of atoms (virus RNA) to hundreds of billions of atoms (DNA). Because nucleic acids are so large, they will statistically have more radiation damage compared to more minor inorganic compounds, which will only marginally be affected. Once damaged, the molecule remains (such as broken DNA strands) are still available for scientific study, but will be sterilized due to the strand damage. Smaller and more stable molecules typically relevant to geology will only be sparsely affected and the sample's chemical makeup will generally be unaffected by ionizing radiation on the order of 100 kGy. Another benefit of radiation sterilization is that the temperature of the sample may not be greatly affected. The radiation source will not appreciably raise the temperature of the sample, and thermally unstable compounds will remain unaffected. Magnetic fields are also largely unaffected by gamma radiation up to several 10's of MGy (over 100 times that of the 100 kGy proposed for biological sterilization). Overall, a low dose rate of ionizing radiation is a minimally invasive sterilization technique. It may work inside of an enclosed volume.

### Radioisotope Selection

Calculations indicate that a sustained dose rate of about 1 mGy/s may be sufficient to produce a total radiation dose of 100 kGy (10 MRad) over three years (an example of an expected time that Mars samples will be in transit in an Earth Return Orbiter).

A dose rate of about 1 mGy/s would correspond to a power level of 1 mW/kg of radiation energy in a perfectly efficient situation. However, the real efficiency will be reduced due to non-uniform energy deposition caused by self-shielding and the isotropic nature of the source, radioisotope decay, and radiation escape away from the areas of interest for sterilization. Efficiency is defined as the lowest dose rate in the sterilization area of interest divided by the dose rate if all radiation energy was deposited evenly over the entire sample. The efficiency depends on the half-life, radiation spectrum, geometry, and materials of the radioisotope and sample return system.

Using the Mars Sample Return as an example mission, each sample may be approximately 25 grams and is contained inside a 57 g sample container [5]. A total mass for a return of 12 sample containers would be approximately 1 kg. Assuming an efficiency of 2.5 percent (a conservative value), a source of no more than 40 mW of radiation power should provide adequate power to ensure a dose rate of about 1 mGy/s across the entire system. This is very small in comparison to traditional NASA Pu-238-based sources. A radioisotope heater unit (RHU) produces 1 W, and a Multi-Mission Radioisotope Thermal Generator (MMRTG) produces 2000 W.

I have considered several factors (criteria) when selecting a radionuclide for in-situ sample return sterilization.

Half-Life: A radioisotope with a half-life on the order of the mission length is necessary to ensure it does not decay before it can irradiate the sample. A radioisotope with a relatively short half-life is also preferable, as the shorter half-life provides a higher decay rate and power production. There are approximately 40 radioisotopes with half-lives between 3 and 3000 years.

Radiation Type: A radioisotope that emits a significant fraction of the decay energy as penetrating radiation (gamma or high-energy X-rays) in its decay chain is preferable for volumetric sterilization. Alpha particles, spontaneous fission products, and pure beta emitters deposit most of their energy within 10's to 100's of micrometers in solid materials, preventing sterilization through the entire thickness of the return sample. Beta emitters may also be used, as they generate bremsstrahlung photons; however, the efficacy of converting beta decay energy to photons is typically low, requiring additional source strength. For surface sterilization, where sterilization is useful for the outer layers with a thickness of 10's to 100's of micrometers, beta and alpha radiation are attractive; and lower energy photons may also be used.

Mean Free Path: The penetration depth across the photon spectrum is an important metric that drives design criteria. The mean-free path measures an average penetration depth. A mean-free path that is too large allows the radiation to pass through the samples without depositing much of their energy. Conversely, a mean-free path that is too short will over-attenuate in a region close to the source and will not reach the entire depth of the sample.

Availability: The radioisotope should be generally available or easily produced in relevant quantities.

There are several potential candidates for radioisotope materials, but I will discuss Cesium-137 (Cs-137) in the context of a volumetric sterilization mission (rather than a surface sterilization). It has a 30-year half-life and is available in relevant quantities. Cs-137 emits 661.7 keV gamma rays. The gamma ray is emitted 85.1 percent of the time with each decay, after accounting for decay branching of 94.5 percent, and accounting for internal conversion.

| | | |
|---|---|---|
| Cs-137=> | 3a-137m + electron + antineutrino => Ba-137 + | 94.6% |
| | gamma | |
| | 3a-137 + electron + antineutrino | 5.4% |

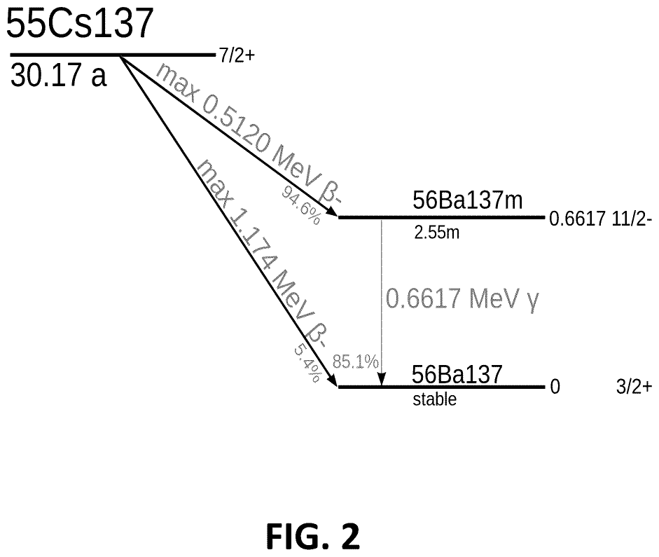

FIG. 2 shows a radioactive decay scheme for Cesium-137 (Cs-137) [6]. The total decay energy for Cs-137 is 1176.5 keV. Approximately 30 percent of that energy is lost in the non-interacting neutrino. Of the remaining 813.8 keV, an average of 565.4 keV is emitted per decay as the 661.7 keV gamma. The augur and beta electrons travel on the order of 1 mm or less, making them unable to reach deep into the sample containers. The electrons interact with the environment and generate secondary Bremmstralauung photons. However, the Bremmstrallaung photon sources are small in number compared to the 661 KeV gamma emission. See Table 1 to see a breakdown of the radiation energy from Cs-137.

TABLE 1
Energy Released from Cs-137.

| Primary | Energy | Energy | |
|---|---|---|---|
| Radiation | [keV/decay] | [%] | Notes |
| Antineutrino | 362.7 | 30.8 | Antineutrino from beta decay does |
| | | | not interact. |
| Beta, Augur | 248.4 | 21.1 | Beta and augur electron travel |
| | | | micrometers. |
| X-Ray | 2.3 | 0.2 | X-rays generated from Augur |
| | | | electron conversion. |
| Gamma | 565.4 | 48.1 | The 661.7 keV gamma |
| Total | 1176.5 | 100 | |

The 661.7 keV gamma ray has a mean-free path that is dependent on the elemental constituency and density of the material being irradiated. Table 2 provides the mean-free path based on XCOM calculations [8].

TABLE 2
Mean Free Path for 661.7 keV gamma in relevant materials.

Material

| | Titanium Metal | Martian Regolith |
|---|---|---|
| | (4.5 g/cm3) | (2.4 g/cm2) [9] |
| Attenuation (g/cm²) | 7.193E−02 | 7.618E−02 |
| Mean Free Path | 3.1 cm | 5.25 cm |

A Cs-137 radioisotope source may be located inside of a sample container. The source may have a diameter of the order of 1-10 centimeters and may be capable of a reasonably uniform energy deposition across the sample container. Assuming a reasonably low 2.5 percent irradiation efficiency results in an initial power of 40 mW. A 40 mW source of 662 keV gammas may require about 11.9 Curies of Cs-137. Accounting for 10 years of decay would require 14.9 Ci on launch for 0.172 grams of Cs-137. The mass of a Cs-137 source would be significantly greater because Cs-137 is typically not isotopically pure and will have decayed based on the specific age of the source. In addition, the radioisotope may be located in a sealed tube known as a “sealed source”, which adds mass to the system. For fresh, fission product-based Cesium, the ratio of Cs-137 to Cs is around 43% [22]. The most common chemical form of Cs-137 is CsCl salt, yielding a specific activity of around 30 Ci/g for fresh sources. Most CsCl sources have decayed by one to two half-lives, reducing the specific activity toward 10 Ci/g [13].

Cs-137 sources have recently been produced in other form factors, such as being vitrified or encapsulated in a glass material. In the last decade, India has scaled a process to produce Cs-137 encapsulated in NaBS glass, which can have a specific activity in the 5-15 Ci/g. In addition, doubly-encapsulated sealed sources using glass obtain a specific activity of around 1.5-4.5 Ci/g [12, 14]. Using a conservative number of 0.5 Ci/g, a 15 Curie sealed radioisotope source may have a mass of about 30 grams.

Today, commercial and government Cs-137 sources are used for dozens of applications, including research irradiators, blood/tissue irradiators, food irradiators, teletherapy sources, brachytherapy, calibration, level gauges, and well logging sources. These sources range from millions of curies to microcuries [15].

A 15 Ci quantity of Cs-137 seems very reasonable regarding availability and mass and is currently available. Even if the system were ten times the mass (e.g., 300 grams), the mass would be feasible within the specified mass requirements outlined for a proposed Mars Sample Return Container.

As stated earlier, there are other potential radioisotopes of interest beyond Cs-137. However, Cs-137 may be a good fit. Other radioisotopes that emit a strong photon energy ranging from about 50 keV to about 1000 keV may be used, as well for volumetric sterilization.

### Sample Return Equipment

The design of a radioisotope-based in-situ sterilization system includes placing radioisotope sources so that the ionizing radiation energy is deposited optimally to the areas of interest. The exact configuration depends on the (Alpha or Beta) particle range or photon spectrum and the mean free path (MFP) of the (Photon) radiation. The sample, and any areas where contamination may be a concern, may be positioned within a few MFP's of the radioisotope source.

In an embodiment, the radioisotope source(s) may be located inside of a sample container.

In a different embodiment, the radioisotope source(s) may be embedded in an Earth return vehicle.

With this innovative in-situ sterilization approach, we can evaluate the big-picture effects on a potential Mars Sample Return (MRS) architecture, for example. By removing complex handling, external sterilization, and sealing systems, the in-situ sterilization approach could reduce the Mars Capture and Containment Return System (MCCRS) mass from about 625 kg to less than or equal to about 100 kg, resulting in an Earth-bound orbiter that is about one half-ton lighter, which would enable a smaller, lower-cost solar array for the same Delta-V for the return to Earth trip.

A second design consideration involves radiation effects on sensitive spacecraft components. The radiation may travel further than necessary and could affect sensitive electronic components. The embodiments disclosed herein may use radiation shielding in the form of shadow shielding, spot shielding, distance shielding, and radiation-hardened electronics to mitigate the effects of radiation on sensitive electronic components. In addition, optimizing the efficiency of the radiation energy to sterilization energy allows for a reduction in the mass of the initial radioisotope source. Different geometrical configurations may also reduce shielding mass, such as placing the radioisotope in the Earth Return Vehicle, rather than in the sample container. A few kg of radiation shielding may be more allowable in the Earth Return Vehicle because it sits in a location in the gravity well of a planetary body where less propellant would be necessary.

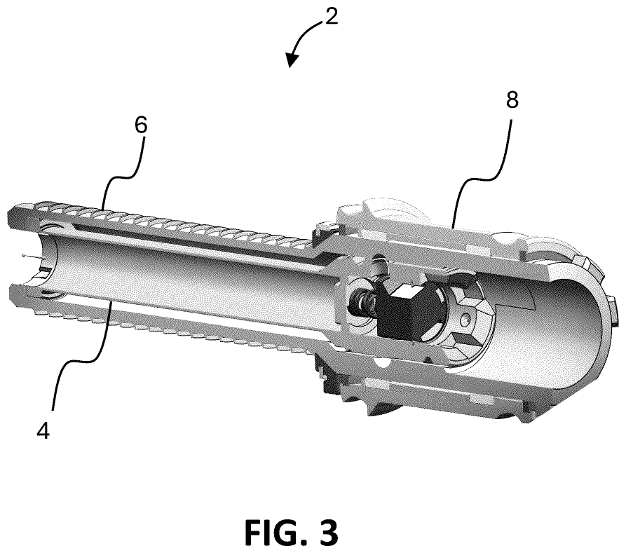

FIG. 3 shows a perspective, 3-D, cut-away view of an example of an individual sample collection tube 2. Sample collection tube 2 includes a sealable, cylindrical sample collection tube 4 covered by an outer protective cylinder 6, both of which may be made of titanium metal of a titanium alloy. Sealing/drill bit drive mechanism 8 is illustrated. Sample collection tube 2 may contain samples of collected planetary soil and/or rock.

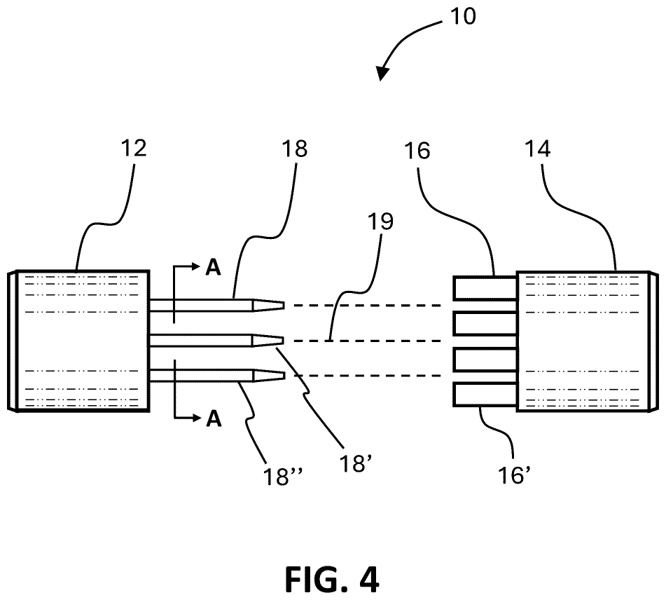

FIG. 4 shows an exploded side view of an example of a sample container 10 with one or more in-situ, radioisotope sterilization sources 18, 18′, etc. and one or more sample collection tubes 16, 16′, etc. contained within sample container 10. Sample container 10 may be hollow and may have a cylindrical outer shape. Sample container 10 may have a pair of mating (concentric), cylindrical cups: proximal cup 12 and distal cup 14, which may be joined together along a common central axis 19 to make a hermetically-sealed, cylindrical sample container 10. Radioisotope source(s) 18, 18′, etc. may be centrally located inside of hollow cylinder 10 and may be disposed adjacent to sample collection tubes 16, 16′ etc.

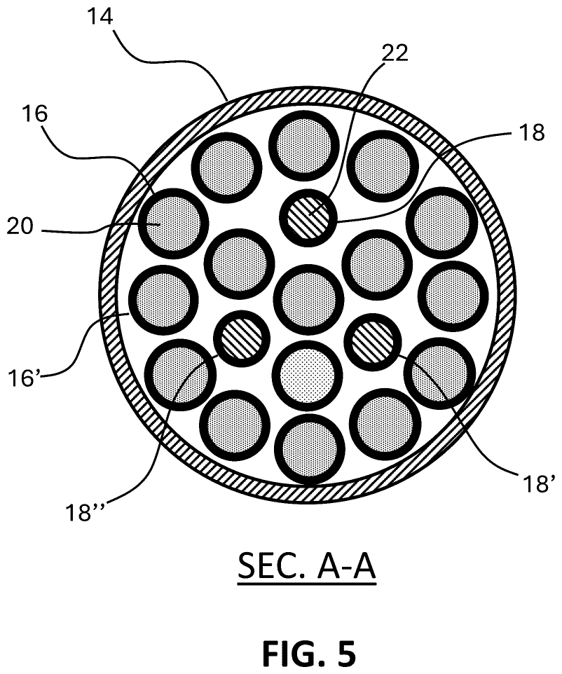

FIG. 5 shows a cross-sectional, end view (SEC. A-A) of the sample container 10 illustrated previously in FIG. 4. In this embodiment, sample container 10 contains three, in-situ, radioisotope sterilization source tubes 18, 18′, etc. and sixteen sample collection tubes 16, 16′, etc., arranged in a close-packed array inside of sample container 10. Planetary sample (soil or rock sample) 20 is sealed within sample collection tube 16, and radioactive radioisotope 22 is sealed within radioisotope source tube 18. Radioisotope source 22 may comprise Cesium-137. Radioisotope source tubes 18, 18′, etc. may be positioned inside sample container 10 in a triangular pattern configured in such a way to optimally irradiate as many adjacent sample collection tubes 16, 16′, etc. as possible (e.g., six sample collection tubes 16, 16′, etc. surrounding an individual radioisotope source tube 18).

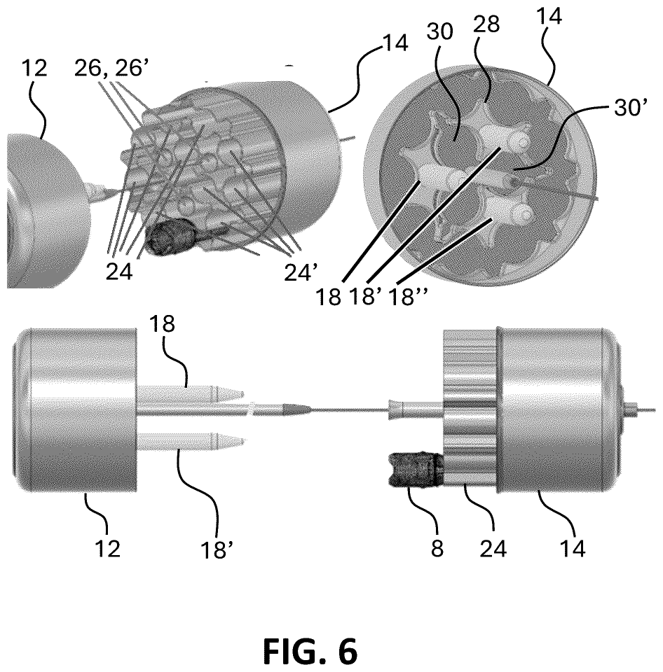

FIG. 6 shows a perspective, 3-D, exploded view of an example of sample container 10 with multiple, in-situ radioisotope source tubes 18, 18′, etc. contained within sample container 10. In this embodiment, sample container 10 includes three radioactive source tubes 18, 18′, etc. disposed in a triangular array, with each radioactive source tube 18, 18′, etc. securely held by a corresponding radioisotope source support structure 26, 26′, etc., respectively. Each sample collection tube 16, 16′, etc. (not shown) may be securely held in a corresponding sample collection tube support structures 24, 24′, etc., respectively. Sample collection tube support structures 24, 24′, etc. may comprise one or more nested, parallel cylinders 24, 24′, etc. (which may be slotted), or a perforated plate 28, with plate 28 having one or more thru-holes 30.

### Radioisotope Regulatory and Safety Issues

One challenge to the use of a radioisotope sterilization system may be the regulatory and launch safety complexity added to the mission by including a radioisotope. In August 2019, the National Presidential Security Memorandum 20 (NPSM-20 [16]) tasked various federal agencies with creating a risk and quantity-based tiered regulatory framework for launching nuclear material. NASA has further codified the regulatory framework with NASA Procedural Requirement Document 8715.26 (NPR 8715.26) [17]. The quantity-based regulatory tiers use an “A2 value” to compare the relative risk of different radioisotopes. The A2 value is the maximum activity of normal-form radioactive material allowed in a Type A package [18].

A 15 Ci Cs-137 source has an A2 value of 0.925. A 60 kCi MMRTG has an A2 value of 2.2 million. A 30 Ci Pu-238 RHU has an A2 value of 1100. Hence, a 30 Ci Cs-137 source is extremely low risk compared to legacy radioisotope missions. The fact that the A2 value is less than 1.0 means there would be a significant simplification of launch safety. NPR 8715.26 Section 4.2 includes a reduced set of required deliverables and approval levels for a launch, including (1) the elimination of the need for doing a nuclear safety analysis, (2) the elimination of a nuclear safety review, (3) the reduction of a launch or reentry request to 3 months before scheduled launch, (4) elimination of NASA general counsel consultation, and (5) elimination from the need to report to the Office of Science and Technology Policy at the White House. The shipment of the radioisotope to the launch facility may be completed in a Type A container, rather than the more complex Type B container. If additional radioisotope material is needed, according to NPR 8415.26 Section 4.2, the next tier above “less than 1×A2” is “1-500×A2” values. Even this level remains a low-burden regulatory pathway.

While radioisotope launch safety must be addressed, the cost and complexity will not be on the scale of existing programs. Also, the licensing burden cost is minor compared to the complexity, and significant mass savings may be enabled by using radioisotope sterilization.

### Surface Decontamination Alternative

An alternative method includes leaving the collected extraterrestrial samples unsterilized in a sealed return container. Then, the sealed return container's outer surface may be sterilized so that the sealed return container may be safely returned to Earth for analysis. The collected sample may ultimately be opened in a biohazard containment facility. The logic behind this alternative option is that this method would maximally preserve the sample(s) for past signs of life or other scientifically interesting information and, hence, enable the study of extant life if it exists. This requirement is often called the “Sample Integrity” requirement [20].

In one embodiment of the Mars Sample Return Mission, about 2 mm of Titanium metal or Titanium alloy forms the outer wall of a Orbiting Sample (OS) container that is roughly 30 cm in diameter and 25 cm in length. The Mars Sample Return Mission is interested in sterilizing small dust particles attached to the OS container's surface, where the dust particles are estimated to be about 20 μm in diameter. According to the present disclosure, the mass of radioisotope required for sterilizing the OS container's surface is much lower than the mass of radioisotope required to perform volumetric sterilization.

In some embodiments, a total surface area may of an OS container be approximately 0.25 square meters. Assuming a 20-μm contamination thickness, then the total volume that requires sterilization may be about 5 cm³, and the total mass of surface contamination may be 1-5 grams (depending on the density assumed for the dust layer).

By carefully considering the type and energy of radiation, radioisotope sources may be optimally selected for sterilizing the outside surface of the OS container.

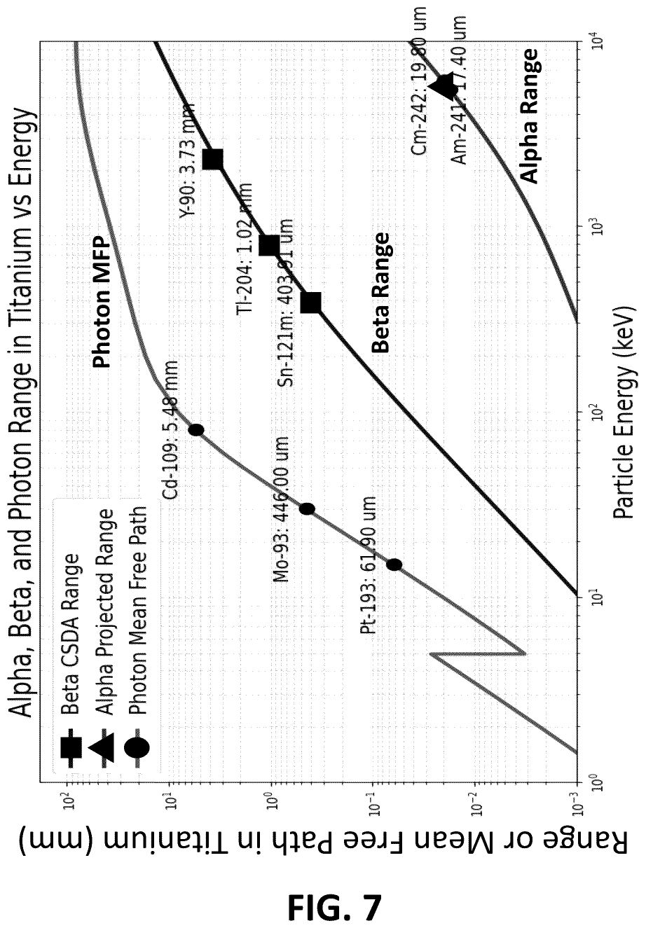

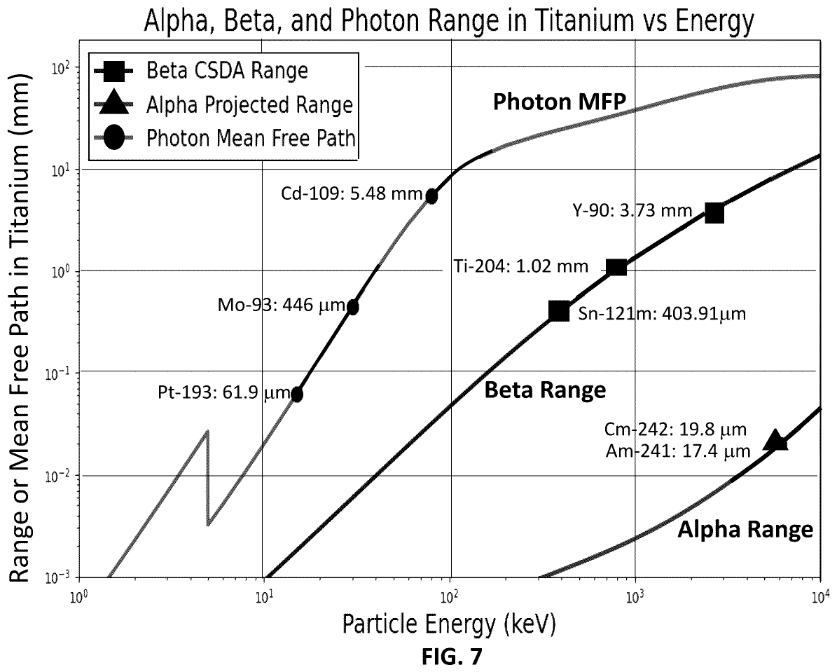

Regarding surface sterilization methods using radioisotope sources, the graphs shown in FIG. 7 illustrates three lines depicting either the Range of particle deposition (mm) in titanium for energetic Alpha or Beta particles, or the Mean Free Path (MFP) deposition distance (mm) in titanium for energetic photons, all as a function of particle (or photon) energy in KeV.

In sterilizing the OS container for a Mars Sample Return, we are looking for a radiation type that will dose only up to 2 mm of Titanium metal wall thickness, and have approximately zero (or at least significantly reduced) dose deposition beyond that thickness. Low-energy photons (e.g., <about 100 KeV); mid-to-high energy Beta particles (e.g., from about 300 KeV to about 2.5 MeV); and high-energy Alpha particles (e.g., greater than about 5 MeV), and combinations thereof, may be used.

Each of these three different radioactive particles/rays has some nuances. For charged particles (Betas and Alphas), they have a defined range. In this case, dose deposition is based on the Continuous Slowing Down Approximation (CSDA) that is defined by the stopping power equation and characterized by a Bragg peak.

For Alpha particles, the energy of the Alpha particle is consistent for every decay, which provide excellent consistency in particle range. In addition, the speed of the Alpha is much lower compared to a Beta of the same energy and, hence, only a minimal amount of Bremsstrahlung radiation will occur. The challenge with Alpha particles, however, is that their deposition range is very short (i.e., less than about 0.4 mm). However, in the case of a Mars Sample Return mission, this may be acceptable. Am-241 is a suitable radioisotope with a 242-year half-life that is routinely used in smoke detectors. Assuming an OS container is entirely coated in 20 μm of soil particles with a density of 2.5 g/cm3, then there would comprise about 12.5 grams of material that need to be sterilized. Assuming a 10 percent efficiency (alpha particles should be higher efficiency than photons or betas due to their emission consistency), a 100 kGy dose delivered over a period of 3 years would require about a 4 mCi source of Am-241. By way of comparison, a standard household smoke detector has an Am-241 activity of roughly 1 μCi. Hence, in this example, a total quantity required for a Mars Sample Return mission would be equivalent to about 4,000 smoke detectors.

For Beta particles, the energy of the Beta is described as a distribution with an average energy equaling approximately one-third of its maximum energy. Beta particles have much less mass and are easily pushed around by electrostatic fields. The CSDA range will overpredict the actual range, depending on the tortuous path that each Beta travels in the material. In addition, Bremsstrahlung radiation is much more intense with Betas than with Alphas because the mass of a Beta electron is much lower. Also, for similar energies, Beta particles will travel much faster. The Bremsstrahlung radiation will typically travel much further than the electron, but it will only carry a small fraction of the energy. Generally, this means that the Beta range data shown in FIG. 7 is affected by both the CSDA and Bremsstrahlung.

Many Beta radioisotopes could be used over a wide range of wall thicknesses ranging from 10's of μm's to a few cm. Looking at, for example, the Tl-204 radioisotope, the CDSA range is around 1 mm in Titanium. However, the effective range is more likely closer to 200 μm. Around 1 Curie of Tl-204 may be effective, but detailed modeling of all of the completing effects may be required. A different radioisotope, Sr-90, shown in FIG. 7 (which decays into Y-90, which itself decays a second time) may be used because of its higher energy of the double-decay process.

Sr-90 is a commonly available isotope with a 30-year half-life. The decay product Y-90 is a deeper penetrator, but after accounting for the shorter range compared to CDSA and the Beta spectrum, 2 mm of Ti may be sufficient to shield the soil and rock samples located inside of the sample container from the external (i.e., surface) radiation. Still, detailed modeling may be necessary to determine the degree to which both electron CSDA and Bremsstrahlung radiation may affect the samples, as there may be at least be a small degree of sample irradiation.

Photons don't have a range per se, but, rather, they have a mean free path (MFP). This means that the photon line in FIG. 4 generally underpredicts the energy deposition range since there is no maximum range and at least some of the photons are likely to make it through the 2 mm thick titanium wall and into the sample. For a photon with a MFP on the order of 200 μm's in titanium, about 10 MFP's may be required to completely penetrate through the Titanium wall and into to the sample. 10 MFP's may reduce the photon flux by about 1000 times, which seems reasonable for respecting the sample's integrity [20].

From this analysis, it is apparent that radiation derived from radioisotope decay may be used to effectively sterilize surfaces. Different radioisotopes may be chosen that offer different attributes useful for various desired surface sterilization configurations and requirements.

In another embodiment, a sterilization method may include applying one or more radioactive coating(s) to the outer surface of an OS container. In some cases, direct uniform deposition of one or more radioisotopes over a large surface area may be accomplished by, for example, electroplating, chemical vapor depositing, physical vapor depositing, cold spraying, and/or 3-D additive printing. The radioisotope(s) may be added to a paint or other bonding material and applied as a thin coating. Alternatively, a thin foil (e.g., gold or copper) with a coating of the radioisotopes(s) may be adhered to, or wrapped around, the OS container's outer surface. Consideration of the thickness of the radioisotope coating and its possible additives needed for adhesion may be required, especially where the sample container's wall thickness is an appreciable fraction of the range of the radioactive particle or ray. In such a case, the surface coating of the radioisotope and possible additives may self-shield the radioisotope's radiation.

### Multi-Type Sterilization

In other embodiments, using various combinations of multiple, different sterilization methods, such as illuminating with ultraviolet (UV), irradiation with Gamma rays, heating to a high temperature, and exposure to toxic chemicals, or combinations thereof, may provide a more robust sterilization method. Combining one or more of these different types of sterilization techniques may be particularly effective because of the following factors:
(A) Unknown Unknowns: We don't understand what forms Martian life may take and there could be some unforeseen resistance or immunity to a specific type of sterilization method that is used.
(B) Strengths and Weaknesses: Different types of sterilization have different strengths, weaknesses, and reliability. While each type may be individually robust, multiple types may be used to avoid possible failure scenarios.
(C) Extra Sterilization: Multiple different types of sterilization method may provide non-linear sterilization benefits. While this may be hard to quantify, an organism being weakened by multiple different sterilization techniques would be stressed in multiple dimensions.
(D) Reduction of Extreme Usage: To assure 100% sterilization, in some cases, a disproportionate amount of additional sterilization may be necessary. For example, with UV light, the penetration distance is small, and a significant amount of extra power may be needed to increase the penetration depth. In the case of radioisotopes, 100 kGy is a conservative radiation dose, and a lower dose might be effective. Combining techniques may reduce the extreme usage of a single method and allow more moderate use of multiple techniques.
(E) Multiple Simultaneous Radioisotopes: Multiple types of different radioisotopes may be used to optimize radiation sterilization, rather than relying on a single species of radioisotope.

Surface sterilization may be used for a Mars Sample Return mission. UV light is one methods proposed for surface sterilization. However, the UV light may not fully penetrate nooks and crannies of surface(s). In this case, combining UV light sterilization with one or more radioisotope(s) sterilization may increase the robustness of sterilization. The UV light may come from the exterior, whereas radioisotope sterilization may come from the interior.

In another embodiment, some metallic surfaces, such as copper or silver surfaces, are known to be antimicrobial. In this case, attaching antimicrobial surface finishes (e.g., copper foil, or electroplated copper or silver surfaces) may provide an additional layer of sterilization.

In another embodiment, three different techniques: (1) an antimicrobial surface finish, (2) UV exterior surface illumination, and (3) radioisotope source(s) attached to the exterior surface, may be used individually, or in combination, to optimize sterilization results.

In another embodiment, an electron accelerator-based system may be used to provide in-situ sterilization.

In an embodiment, a method of sterilization may include:
(a) providing a sample container;
(b) inserting a sample tube, containing a sample, into the sample container;
(c) inserting a radioisotope source into the sample container; and
(d) irradiating the sample with radiation emitted by the radioisotope source.

In an embodiment, a method of surface sterilization may include:
(a) providing a sample container having an outside surface;
(b) providing a radioisotope source that is disposed outside of the outside surface of the sample container; and
(c) irradiating the outside surface of the sample container with radiation emitted by the radioisotope source.

In an embodiment, a method of surface sterilization may include:
(a) providing a sample container having an outside surface, containing one or more samples;
(b) sterilizing the one or more samples contained inside of the sample container;
(c) providing a radioisotope source that is disposed outside of the outside surface of the sample container after sterilization has been completed; and
(d) irradiating the outside surface of the sample container with radiation emitted by the radioisotope source.

In an embodiment, a sterilization apparatus may include: a sample container; a sample collection tube; a sample disposed inside of the sample collection tube; and a radioisotope source; wherein the sample collection tube is disposed inside of the sample container; wherein the radioisotope source is disposed inside of the sample container.

In an embodiment, a sterilization apparatus may include: a sample container having an outside surface; a sample collection tube; a sample disposed inside of the sample collection tube; and a radioisotope source disposed on the outside surface of the sample container; wherein the sample collection tube is disposed inside of the sample container.

In an embodiment, an apparatus may include: a sample container having an outside surface or concentric containers with at least one sealed outside surface; a sample collection tube; a sample disposed inside of the sample collection tube; and a radioisotope source disposed on the outside surface of the sample container; wherein the sample collection tube is disposed inside of the sample container.

All references disclosed herein are incorporated herein by reference in their entirety.

The detailed description and the drawings or figures are supportive and descriptive of the present teachings, but the scope of the present teachings is defined solely by the claims. While some of the best modes and other embodiments for carrying out the present teachings have been described in detail, various alternative designs and embodiments exist for practicing the present teachings defined in the appended claims. All embodiments and examples disclosed herein are non-limiting embodiments and non-limiting examples. The words “a”, “an”, “the”, “at least one”, and “one or more” are used interchangeably to indicate that at least one of the items is present.

### REFERENCES

[1] Singleton, E. V, David, S. C., Davies, J. B., Hirst, T. R., Paton, J. C., Beard, M. R., Hemmatzadeh, F., & Alsharifi, M. (2020). Sterility of gamma-irradiated pathogens: a new mathematical formula to calculate sterilizing doses. Journal of radiation research, 61(6), 886-894. https://doi.org/10.1093/jrr/rraa076
[2] Tallentire, A. (1980). The spectrum of microbial radiation sensitivity. Radiation Physics and Chemistry (1977), 15(1), 83-89. doi:10.1016/0146-5724(80)90101-6
[3] Hanson, J et. al., “Radiation Sterilization: Dose is Dose” Biomedical Instrumentation & Technology, Volume 54, pg. 45-52, 2020
[4] Kowalski, J., & Tallentire, A. (1999). Substantiation of 25 kGy as a sterilization dose: A rational approach to establishing verification dose. Radiation Physics and Chemistry, 54(1), 55-64. https://doi.org/10.1016/S0969-806X(98)00206-0
[5] National Aeronautics and Space Administration (Accessed 2024, May 14th). A Martian roundtrip: NASA's Perseverance rover sample tubes. 2020 https://www.nasa.gov/centers-and-facilities/jpl/a-martian-roundtrip-nasas-perseverance-rover-sample-tubes/
[6] Caesium-137. (accessed 2024, April 14). In Wikipedia. https://en.wikipedia.org/wiki/Caesium-137
[7] NNDC, et al., Evaluated Nuclear Structure Data File (ENSDF). United States: N. p., 2022. Web. doi:10.18139/nndc.ensdf/1845010.
[8] M. J. Berger et. Al. “NIST Standard Reference Database 8 (XGAM),” NIST https://dx.doi.org/10.18434/T48G6X, 2010
[9] R. Rieder et. al., “Chemistry of Rocks and Soils at Meridiani Planum from the Alpha Particle X-ray Spectrometer,” Science, New Series, vol. 306, no. 5702.
[10] G. L. Tingey et. al. “A review of Safety Issues that Pertain to the use of WESF Cesium Chloride Capsules in an Irradiator,” Dept of Energy, Tech Report, PNL-5170, 1984
[11] S. Goodwin et. al., “Current Landscape of Alternative Technologies for Calibration Applications”, Institute of Nuclear Materials Management, 2023
[12] Diwan, J., Dharashiokar, A., Wadhwa, S., Kumar, A., Tomar, N. S., & Kaushik, C. P. (2022, January-February). Challenges to innovations: Industrial scale production of cesium glass pencils for blood irradiators. BARC Newsletter, 42-44.
[13] L. Price, “Cs-135 Content of Cesium Capsules and Strontium/Cesium Heat Sources-18509,” WM2018 Conference, Mar. 18-22, 2018, Phoenix, Arizona, USA, 2018
[14]“Design & Development of Facility for Production of Active Cs-137 Source Pencils for Blood Irradiator.” November-December 2015 BARC Newsletter, p. 56-63. Bhabha Atomic Research Centre, Technology Development Division, Mumbai & Homi Bhabha National Institute, Mumbai., 2015
[15]“Categorization of Radioactive Sources,” Safety Guide No. RS-G-1.9, International Atomic Energy Agency, 2005
[16] The White House, “National Security Presidential Memorandum-20 (NSPM-20),” 2019
[17] NASA, NASA Procedural Requirement 8715.26, 2022
[18]“Specific Safety Requirements No. SSR-6 (Rev. 1), Regulations for the Safe Transport of Radioactive Material 2018 Edition,” International Atomic Energy Agency, 2018
[19] Miekka S I, Forng R Y, Rohwer R G, MacAuley C, Stafford R E, Flack S L, MacPhee M, Kent R S, Drohan W N. Inactivation of viral and prion pathogens by gamma-irradiation under conditions that maintain the integrity of human albumin. Vox Sang. 2003 January; 84(1):36-44. doi: 10.1046/j.1423-0410.2003.00256.x. PMID: 12542732.
[20] Wadhwa, M., Carrier, B. L., & Goreva, Y S. (2022). Sample science considerations for Mars Sample Return. 53rd Lunar and Planetary Science Conference, Abstract #2717.
[21] Samin A., “A review of radiation-induced demagnetization of permanent magnets,” Journal of Nuclear Materials, Volume 503, 2018, Pages 42-55.
[22] Wieser, A., Lachner, J., Martschini, M., Zok, D., Priller, A., Steier, P., & Golser, R. (2023). Detection of 135Cs & 137Cs in environmental samples by AMS. Nuclear Instruments and Methods in Physics Research Section B: Beam Interactions with Materials and Atoms, 541, 1-7.

What is claimed is:

1. A method, comprising:
(a) providing a sample container;
(b) inserting a sample collection tube, containing a sample, into the sample container;
(c) providing a radioisotope source; and
(d) irradiating the sample with radiation emitted by the radioisotope source.

2. The method of claim 1, step (d) comprises irradiating the sample with ionizing radiation at a dose rate of at least 0.001 Gy/s.

3. The method of claim 1, wherein step (d) comprises irradiating the sample to a total dose of at least 100 kGy.

4. The method of claim 1, wherein the radioisotope source comprises Cesium-137.

5. The method of claim 1, wherein the radioisotope source is located inside of the sample container.

6. The method of claim 1, wherein the radioisotope source is located outside of the sample container.

7. The method of claim 1, wherein the radioisotope source radiates photons with a photon energy ranging from about 50 keV to about 1000 keV.

8. The method of claim 1, further comprising irradiating the sample to a dose sufficient to achieve a Sterilization Assurance Level for microbes that ranges between about 10⁻¹² to about 10⁻²⁴.

9. A method, comprising:
(a) providing a sample container having an outside surface;
(b) providing a radioisotope source that is disposed outside of the outside surface of the sample container; and
(c) irradiating the outside surface of the sample container with radiation emitted by the radioisotope source.

10. The method of claim 9, wherein the radioisotope source emits at least one type of radiation selected from the group consisting of low energy photons having an energy less than 100 keV, Beta particles having an energy ranging from about 300 keV to about 2.5 MeV, and Alpha particles having an energy greater than about 4 MeV, and combinations thereof.

11. The method of claim 9, wherein the radioisotope source comprises at least 4 mCi of Am-241 or at least 1 Ci of Tl-204.

12. The method of claim 9, wherein the radioisotope source emits photons having a mean free path of less than or equal to about 200 μm in Titanium.

13. The method of claim 9, further comprising applying a radioactive coating to the outside surface; wherein the radioactive coating comprises the radioisotope source.

14. The method of claim 13, wherein applying the radioactive coating comprises: electroplating, chemical vapor deposition, physical vapor deposition, cold spraying, or 3-D additive printing the radioisotope source onto the outside surface, or combinations thereof.

15. The method of claim 13, wherein applying the radioactive coating comprises adding the radioisotope source to a paint and then depositing a radioactive paint to the outside surface.

16. The method of claim 9, further comprising treating the outside surface with at least two different sterilization methods selected from the group consisting of: illuminating with ultraviolet light, irradiating with Gamma rays, heating to an elevated temperature, and applying a toxic chemical.

17. The method of claim 9, further comprising subjecting the outside surface of the sample container to a combination of three different sterilization methods, comprising:
(1) using an antimicrobial surface finish;
(2) illuminating the outside surface with ultraviolet light, and
(3) irradiating the outside surface with radiation emitted by one or more radioisotope sources attached to the outside surface.

18. An apparatus, comprising:
a sample container;
a sample collection tube;
a sample disposed inside of the sample collection tube; and
a radioisotope source;
wherein the sample collection tube is disposed inside of the sample container; and
wherein the radioisotope source is disposed inside of the sample container.

19. The apparatus of claim 18,
wherein the sample container comprises a hollow cylinder; and
wherein the radioisotope source is centrally located inside of the hollow cylinder and is disposed adjacent to the sample collection tube.

20. The apparatus of claim 19,
wherein the sample container comprises a proximal cylindrical cup and a distal cylindrical cup configured to mate together along a common central axis to form a sealed sample container;
wherein the radioisotope source is attached to the proximal cylindrical cup; and
wherein the sample collection tube is disposed inside of the distal cylindrical cup.

21. The apparatus of claim 18,
further comprising a sealed cylindrical radioisotope source tube containing the radioisotope source;
wherein the sample container comprises a radioisotope source support structure configured for holding the sealed cylindrical radioisotope source tube inside of the sample container; and
wherein the sample container further comprises a sample collection tube support structure configured for holding the sample collection tube inside of the sample container.

22. The apparatus of claim 18, wherein the radioisotope source comprises Cesium-137.

23. An apparatus, comprising:
a sample container having an outside surface;
a sample collection tube;
a sample disposed inside of the sample collection tube; and
a radioisotope source disposed outside of the sample container; and
wherein the sample collection tube is disposed inside of the sample container.

24. The apparatus of claim 23, wherein the radioisotope source emits at least one type of radiation selected from the group consisting of low energy photons with an energy less than 100 KeV, Beta particles with an energy ranging from about 300 KeV to about 2.5 MeV, and Alpha particles with an energy greater than about 5 MeV, and combinations thereof.

25. The apparatus of claim 23, wherein the radioisotope source comprises at least 4 mCi of Am-241 or at least 1 Ci of Tl-204.

26. The apparatus of claim 23,
wherein the sample container comprises titanium; and
wherein the radioisotope source emits photons with a mean free path of less than or equal to about 200 μm in Titanium.

27. The apparatus of claim 23, wherein the outside surface of the sample container is coated with metallic copper and/or metallic silver to provide a layer of antimicrobial sterilization.
